(19) United States
(12) Patent Application Publication
(10) Pub. No.: US 2013/0079566 A1
(43) Pub. Date: Mar. 28, 2013

(54) CATALYTIC PROCESS FOR CONVERSION OF BIOMASS INTO HYDROCARBON FUELS

(71) Applicant: Board of Regents of the Nevada System of Higher Education, on behalf of the University of Nevada, Reno, Reno, NV (US)

(72) Inventor: Hongfei Lin, Reno, NV (US)

(73) Assignees: Nevada,, Reno, NV (US); Board of Regents of the Nevada System of Higher Education, on behalf of the University of

(21) Appl. No.: 13/628,656

(22) Filed: Sep. 27, 2012

Related U.S. Application Data
(60) Provisional application No. 61/539,649, filed on Sep. 27, 2011.

(51) Int. Cl. C10G 1/04; C07C 1/22

(52) U.S. Cl. 585/242; 585/240; 585/310

(57) ABSTRACT

A process for the conversion of lignocellulosic biomass to hydrocarbons is provided. The biomass is subjected to aqueous phase partial oxidation (APPO) in the presence of a heterogeneous oxidation catalyst to selectively provide one or more carboxylic acids in good yields. The carboxylic acids are further upgraded to hydrocarbons in the presence of one or more catalysts, which are capable of catalyzing a ketonization reaction, an aldol condensation reaction, a hydrodeoxygenation reaction, or combinations thereof, and then separating out the hydrocarbons from the one or more catalysts.

### RELATED APPLICATIONS

This application claims the benefit of U.S. Provisional Application Ser. No. 61/539,649, filed on Sep. 27, 2011, which is hereby incorporated by reference herein in its entirety.

### TECHNICAL FIELD

The presently disclosed subject matter relates to processes and systems for converting biomass to platform organic acid products that can be converted to biofuels, which can be replacements for transportation fuels such as gasoline, diesel, and jet fuel.

### BACKGROUND

The use of biomass feedstocks for production of biofuels is motivated by both economic and environmental concerns, including reduction of greenhouse gas emissions, enhancement of the fuel supply, and maintenance of the rural economy. Biomass provides a large renewable source of potential starting materials for the production of a variety of chemicals, plastics, fuels and feeds. For example, biomass feedstocks comprise a variety of carbohydrate-containing biopolymers, such as cellulose, hemicellulose, and pectin, which can be hydrolyzed to provide sugars for use in the fermentation production of alcohol fuels, such as ethanol, methanol, and butanol. However, the conversion of lignocellulosic biomass to fermentable sugars is still cost prohibitive.

Generally, the complex structure of various biomass materials, such as lignocellulosic materials, calls for some form of thermochemical intervention or enzymatic pretreatment to increase saccharification of the carbohydrates. The major thermochemical conversion technologies include gasification, pyrolysis, hydrolysis, and hydrothermal liquefaction. Gasification is a relatively mature and energy intensive process operated at very high temperatures, e.g., 700° C.-900° C., to convert biomass into syngas, which in turn can be further converted to liquid fuels through the Fischer-Tropsch process. Pyrolysis produces a complex mixture of oxygenates as the pyrolysis oil, which requires extensive upgrading, as well as a significant amount of char. Both gasification and pyrolysis require a drying and grinding pretreatment of biomass, which would levy a high energy penalty since the moisture contents of most biomass can be as high as 50% per dry weight of biomass.

Hydrolysis and hydrothermal liquefaction, on the other hand, are operated in aqueous phase and thus can use wet biomass. However, hydrolysis only utilizes the carbohydrate (e.g., cellulose and/or hemicellulose) portion of the biomass, leaving lignin untreated and low value by-product. Mineral acids, which are widely used as the homogeneous catalyst in the hydrolysis process, also raise the environmental concern since disposal of waste mineral acid streams is environmentally problematic. Hydrothermal liquefaction is operated in high temperature water (HTW) at medium to high temperatures and high pressures to yield crude bio-oil or syngas. The drawback is the need for specialized materials to withstand high pressure and corrosive hydrothermal media as the temperature is close to or higher than the critical point of water, i.e., 374° C. Further, the abovementioned processes are normally operated under inert atmosphere. Thus, the high capital and operation costs, as well as the low-yield of desired products hinder the commercialization of these current thermochemical conversion technologies.

Thus, there is a continuing need for improved processes and systems for converting biomass to platform organic acid products, which are suitable for converting into biofuels and other useful products.

### SUMMARY

Certain aspects of the present disclosure are described in the appended claims. There are additional features and advantages of the subject matter described herein. They will become apparent as this specification proceeds. In this regard, it is to be understood that the claims serve as a brief summary of varying aspects of the subject matter described herein. The various features described in the claims and below for various embodiments may be used in combination or separately. For example, specified ranges may be inclusive of their recited endpoints, unless explicitly excluded. Any particular embodiment need not provide all features noted above, nor solve all problems or address all issues noted above.

According to embodiments of the present invention, a flexible, environmentally sound process and system for producing biomass-derived chemicals, such as fuels and/or fuel blends, is provided that generates product streams that can be readily and flexibly adapted to different biomass feedstocks, and may produce different mixtures of renewable products based on market demand. The process and system can also advantageously provide product streams having well-defined, predictable chemical compositions.

According to one embodiment, a process for converting a biomass into hydrocarbons is provided. The process includes a) reacting the biomass under aqueous phase partial oxidation conditions in the presence of a heterogeneous oxidation catalyst to convert greater than 60 weight percent of the biomass to one or more carboxylic acids; b) upgrading the one or more carboxylic acids to hydrocarbons in the presence of one or more catalysts, the catalysts catalyzing a ketonization reaction, an aldol condensation reaction, a hydrodeoxygenation reaction, or combinations thereof; and c) separating out hydrocarbons from the one or more catalysts.

According to another embodiment, a process for converting lignocellulosic biomass into hydrocarbons is provided. The process includes a) reacting the lignocellulosic biomass under aqueous phase partial oxidation conditions in the presence of a heterogeneous oxidation catalyst and a reactive gas to convert greater than 60 weight percent of the lignocellulosic biomass to a plurality of carboxylic acids, wherein the plurality of carboxylic acids includes at least levulinic acid or lactic acid; b) upgrading the plurality of carboxylic acids to hydrocarbons in the presence of one or more catalysts, the catalysts catalyzing a ketonization reaction, an aldol condensation reaction, a hydrodeoxygenation reaction, or combinations thereof; and c) separating out hydrocarbons from the one or more catalysts.

### BRIEF DESCRIPTION OF THE DRAWINGS

The accompanying drawings, which are incorporated in and constitute a part of this specification, illustrate embodiments of the invention and, together with a general description of the invention given above, and the detailed description of the embodiments given below, serve to explain the principles of the invention.

FIG. 1 is a schematic depiction of an integrated biorefinery combining a distributed biomass deconstruction process via an aqueous phase partial oxidation (APPO) route with a centralized platform intermediates upgrading process according to one embodiment of the invention;

FIG. 2 is an illustration of the APPO of cellulose to produce carboxylic acids;

FIG. 3 is an illustration of the ketonization of two acetic acid molecules (C2) to produce acetone (C3) and complementary pathways to C6 products;

FIG. 4 is an illustration of the hydrogenation of acetic acid (C2) to produce ethylene (C2), which undergoes C—C coupling upgrading with three ethylene molecules to form a C6 alkene;

FIG. 5 is a reaction schematic for upgrading lactic acid over 0.1% Pt/Nb₂O₅ catalyst, according to another embodiment of the invention;

FIG. 6 is a reaction schematic for upgrading levulinic acid, according to another embodiment of the invention;

FIG. 7 is a schematic depiction of an integrated biorefinery via the APPO process according to another embodiment of the invention;

FIG. 8 is a graphical representation showing the effects of O₂ partial pressure on the APPO processing of xylan with ZrO₂ catalyst according to another embodiment of the invention;

FIG. 9 is a graphical representation showing the effects of temperature on the APPO processing of cellulose according to another embodiment of the invention;

FIG. 10 is an overlay of GC/MS spectra relating to the APPO conversion of hemicellulose (e.g., xylan) according to another embodiment of the invention; and

FIG. 11 is a GC/MS spectrum of aqueous phase products derived from the APPO processing of lignin with 10% MgO/ZrO₂ catalyst according to another embodiment of the invention.

### DETAILED DESCRIPTION OF EMBODIMENTS

Unless otherwise explained, all technical and scientific terms used herein have the same meaning as commonly understood by one of ordinary skill in the art to which this disclosure belongs. In case of conflict, the present specification, including explanations of terms, will control. The singular terms “a,” “an,” and “the” include plural referents unless context clearly indicates otherwise. Similarly, the word “or” is intended to include “and” unless the context clearly indicates otherwise. The term “comprising” means “including;” hence, “comprising A or B” means including A or B, as well as A and B together.

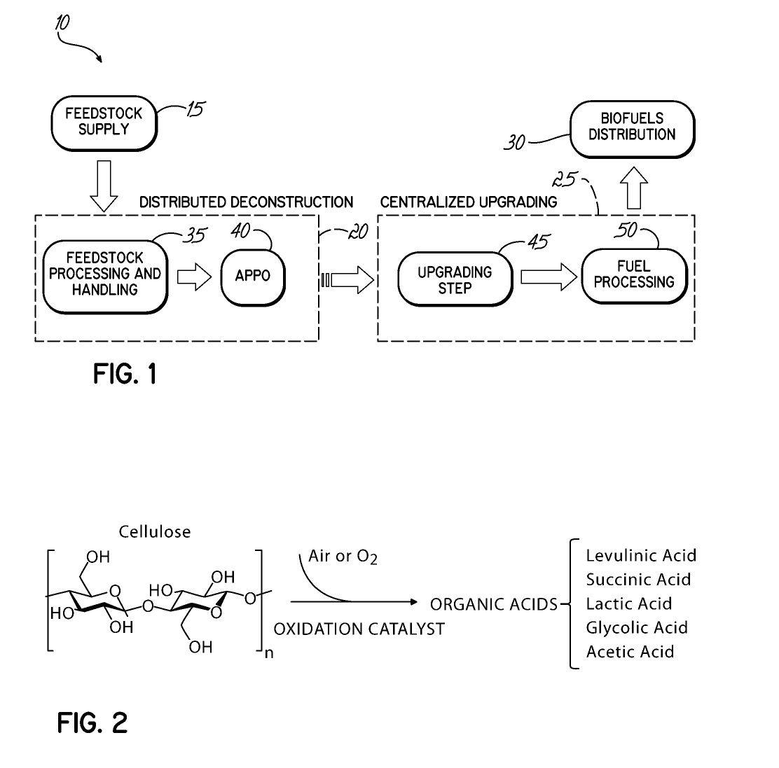

Embodiments of the present invention are directed to the conversion of biomass, such as lignocellulosic biomass, to biomass-derived chemicals, fuels and/or fuel blends that are useful as substitutes or supplements for oil-based chemicals. According to one embodiment, a process for producing hydrocarbons from biomass is provided. This process may be utilized in an integrated biorefinery, such as that schematically represented in FIG. 1, to produce biofuels.

As shown in FIG. 1, the integrated biorefinery 10 generally entails a feedstock supply 15, a distributed deconstruction phase 20, a centralized upgrading phase 25, and biofuels distribution 30. More specifically, the biomass conversion to fuel begins with a feedstock supply 15 of lignocellulosic biomass, for example, which can first undergo feedstock processing and handling 35 prior to being transferred to a thermochemical treatment vessel and subjected to aqueous phase partial oxidation (APPO) processing 40. For example, the biomass can be milled prior to undergoing APPO processing. Under the appropriate APPO conditions, as discussed further below, the lignocellulosic biomass is deconstructed into a variety of platform carboxylic acids, such as monocarboxylic acids, hydroxy-carboxylic acids, dicarbocylic acids, etc. These platform carboxylic acids then move from the distributed deconstruction phase 20 to the centralized upgrading phase 25 whereat they first undergo an upgrading step 45 to form hydrocarbons in the presence of one or more catalysts, which are capable of catalyzing a ketonization reaction, an aldol condensation reaction, and/or a hydrodeoxygenation reaction. These hydrocarbons can be separated from the reaction mixture, which includes the one or more catalysts, using standard separation/purification techniques in a fuel processing step 50 to provide a biofuel that is ready for biofuels distribution 30.

One aspect of producing biomass-derived chemicals, fuels, and/or fuel blends is selecting a source of biomass material. As the most abundant carbon source on earth, there are hundreds of thousands of different plant biomass species. Despite the species variation, the common basic building blocks of plant biomass are the same and include carbohydrates, lignin, protein, and lipids. These building blocks are assembled to rigid and robust biopolymers or large biomolecules to support the growth of plant biomass. For example, lignocellulosic biomass includes cross-linked biopolymers such as cellulose, hemicellulose, pectin, and lignin. And depending on the source of the lignocellulosic biomass, it may include, for example, about 35% to about 50% cellulose, about 25% to about 35% hemicelluloses, and about 15% to about 30% lignin. While from a commercial scale perspective, a sufficient and secure supply of inexpensive and high quality biomass feedstocks is critical, the processes described herein are not particularly limited to any specific source of biomass source. Suitable biomass sources may include, but are not limited to, a woody biomass such as loblolly pine and Douglas-fir, agricultural waste such as corn stover, rice straw, and sugarcane bagasse, marginal farmland crop such as camelina, switchgrass, and Curlytop Gumweed (grindelia squarrosa), or algae from aquatic sources such as Giant Kelp (macrocystis pyrifera).

As used herein, the aqueous phase partial oxidation (APPO) process refers to an oxidative depolymerization process for cleaving biomass, such as lignocellulosic biomass, by in situ generated oxidants, such as hydroxyl radicals and/or superoxide radicals, over one or more oxidation catalysts in an aqueous medium to provide carboxylic acid moieties. According to one aspect, the APPO process maximizes the carbon yield of water soluble carboxylic acid molecules, which can be further upgraded to fuel components or chemicals, by depolymerizing solid biomass under mild oxidative conditions.

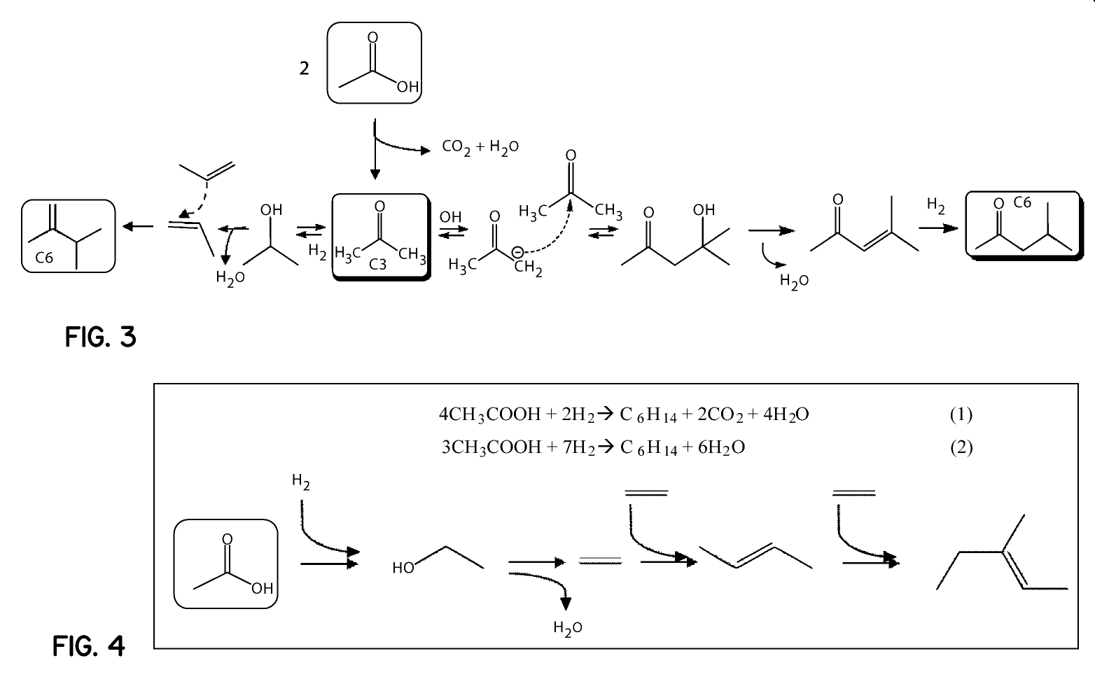

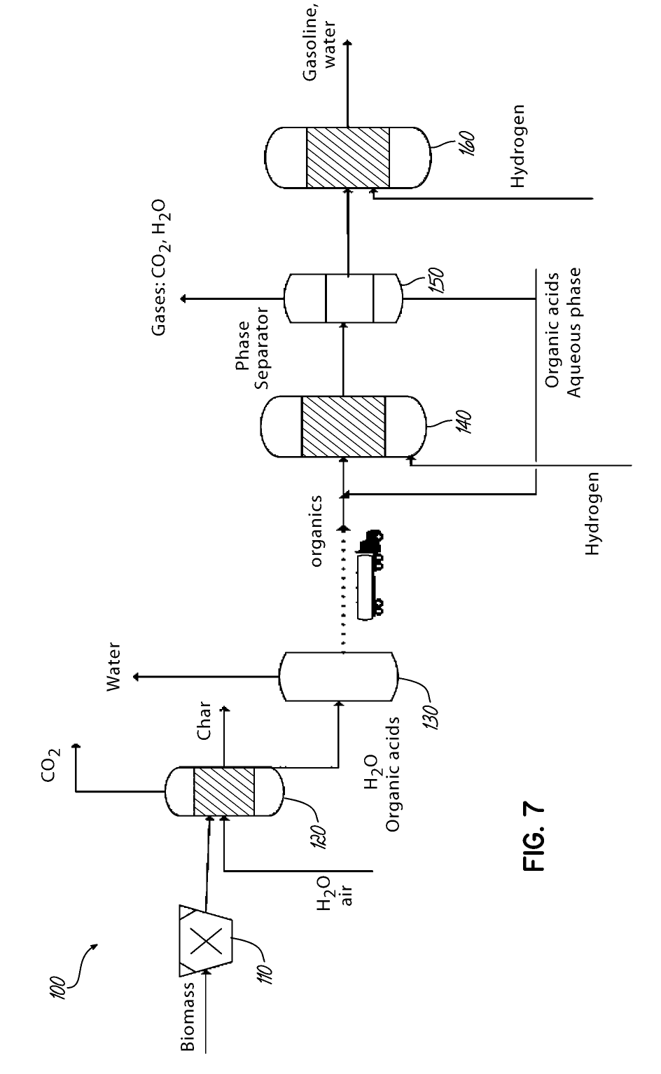

Without being bound by any particular theory, it is hypothesized that the APPO process includes a catalytic generation of hydroxyl radicals or superoxide radicals, which firstly hydrolyze and/or oxidize the solid biomass material to generate water soluble sugar acids, oligosaccharides, phenolics, etc; and these intermediates are further oxidized over the catalyst(s) into more stable carboxylic acids. Catalytic oxidation of the monosaccharide building blocks of cellulose or hemicellulose in aqueous media can generate C2 to C6 carboxylic acids with proper manipulation of catalyst selectivity. On the other hand, lignin, a refractory biopolymer towards hydrolysis and pyrolysis, is susceptible to oxidative cracking. The lignin oxidation products range from aromatic aldehydes to carboxylic acids. In short, all major components of lignocellulosic biomass, hemicellulose, cellulose, and lignin, can be converted into carboxylic acids through the APPO process. As shown in FIG. 2, cellulose can be converted into a variety of platform carboxylic acids, including levulinic acid, succinic acid, lactic acid, glycolic acid, acetic acid, etc. As shown in FIGS. 3-6, these organic acids can be further upgraded into liquid hydrocarbon fuels and value added chemicals. And as shown in FIG. 7, an integrated biorefinery 100 can be provided for the conversion of biomass, such as lignocellulosic biomass, to gasoline via the APPO process described herein.

The APPO process has inherent merits of desirable recovery and reuse of heterogeneous oxidation catalysts and only requires a reactive gas, such as oxygen, and water as the reaction agents and solvents. The instant APPO process takes advantage of various characteristics, which includes the following: (i) the partial oxidation is an exothermic reaction, thereby allowing the reaction heat to assist with off-setting the external energy input required to heat the aqueous reaction mixture to the reaction temperature; (ii) no inert atmosphere or reducing atmosphere (e.g., hydrogen) is needed for the process gas; (iii) heterogeneous oxidation catalysts are more robust (i.e., more tolerant to impurities) as compared to traditional hydrogenation catalysts, and the lifetime of the heterogeneous oxidation catalysts exceeds that of traditional hydrogenation catalysts; (iv) char formation is much lower than pyrolysis processes; (v) it can be used for all types of solid biomass and can be more easily adapted to changes in feedstock composition than alternative approaches; and (vi) it is an environmentally friendly liquid phase process with no emission of air pollutants such as SOx, NOx, and VOC.

Suitable heterogeneous oxidation catalysts can include, but are not limited to, transition metals such as platinum, palladium, ruthenium, rhenium, rhodium, iridium, gold, silver, copper, nickel and cobalt, as well as bimetallic and tri-metallic combinations thereof; metal oxides such as zirconium oxide, titanium dioxide, cerium oxide, vanadium oxide, scandium oxide, manganese oxide, chromium oxide, cobalt oxide, iron oxide, nickel oxide, ruthenium oxide, niobium oxide, tantalum oxide, molybdenum oxide, tungsten oxide, rhenium oxide, lanthanum oxide, copper oxide, zinc oxide, calcium oxide, strontium oxide, barium oxide, and magnesium oxide, and/or mixed metal oxides such as ZSM-5, Zeolite β, Zeolite Y, tungstate zirconia, lanthanate zirconia, ceria zirconia, and hydrotalcite.

Heterogeneous oxidation catalysts may also include a variety of metal oxides such as ZrO₂, TiO₂, CeO₂, Sc₂O₃, V₂O₅, MnO₂, Cr₂O₃, Co₃O₄, Fe₂O₃, NiO, RuO₂, Ta₂O₅, ReOₓ, WOₓ, MoOₓ, Nb₂O₅, La₂O₃, ZnO, CuO, CaO, SrO, BaO, and/or MgO. Heterogeneous oxidation catalysts may include a variety of mixed oxides of the abovementioned metal oxides, e.g., TiO₂—ZrO₂, CeO₂—ZrO₂, CoO/ZrO₂, V₂O₅—ZrO₂, WOₓ—ZrO₂, MoO₂—ZrO₂, MoO₃—ZrO₂, Nb₂O₅—ZrO₂, La₂O₃—ZrO₂, MgO—ZrO₂, CrOₓ/ZrO₂, MnOₓ/ZrO₂, CuO/ZrO₂, WO/ZrO₂, MoOₓ/ZrO₂, VOₓ/ZrO₂, etc. Moreover, heterogeneous oxidation catalysts may include a combination of abovementioned transition metal loaded metal oxides, e.g. Pt/ZrO₂, Pd/ZrO₂, Au/ZrO₂, Pt/Re/ZrO₂, Pt/Rh/ZrO₂, etc. Furthermore, heterogeneous oxidation catalysts may include SiO₂, Al₂O₃, and SiO₂—Al₂O₃ supported metal oxides or metal catalysts. SiO₂ and Al₂O₃ based supports can include amorphous SiO₂—Al₂O₃ and a variety of zeolites and mesoporous materials, such as ZSM-5, Zeolite X, Zeolite Y, Zeolite β, Mordenite, Ferrierite, AlPO-36, AlPO-5, MCM-22, TS-1, etc. Exemplary APPO catalysts include ZrO₂, Au on ZnO, Au on ZrO₂, Au on TiO₂, Au on γ-Al₂O₃, Au on α-Al₂O₃, Au on zeolite-Y, zeolite-β, ZSM 5, Zeolite-Y, or combinations thereof.

Further, according to another aspect of the invention, the heterogeneous oxidation catalyst may also be modified to alter the acidity or basicity of the catalyst surface. According to one embodiment, the heterogeneous oxidation catalyst may be modified by impregnating with a Brønsted acid or base to increase the surface Brønsted acidity or basicity, respectively. For example, zirconium oxide may be treated with sulfuric acid (H₂SO₄) or sodium hydroxide (NaOH) to provide a sulfated zirconium oxide or a sodium hydroxide passivated zirconium oxide.

Moreover, according to another aspect, the efficiency of the heterogeneous oxidation catalysts to activate the reactive gas (e.g., oxygen) can be related to its structure and specific surface area. For example, zirconium oxide displays three polymorphs: monoclinic, tetragonal, and cubic. To further investigate the relationship between catalyst structure and the reactivity towards the APPO of cellulose, both the monoclinic ZrO₂ (m-ZrO₂) and the tetragonal ZrO₂ (t-ZrO₂) with the BET surface areas of 117 m²/g and 108 m²/g, respectively, were evaluated under the same reaction conditions. The surface morphology of the two types of ZrO₂ catalysts were characterized by scanning electron microscopy, which showed different porous structures for the two types. Further, X-ray diffraction (XRD) patterns of both catalysts also indicated no mixed phases in either ZrO₂ material. Under the same APPO conditions, the yield of levulinic acid was 42% over the m-ZrO₂ but decreased dramatically to 12% on the t-ZrO₂. The significant difference in the yield of levulinic indicates that there is a strong connection between the catalyst structure and the surface reaction activity. Monoclinic ZrO₂ was found to form stronger bonds with CO₂ and CO and have stronger Lewis acidity than t-ZrO₂, which may promote the decarboxylation and dehydration reactions and facilitate the conversion of gluconic acid to levulinic acid. Monoclinic ZrO₂ maintained high stability against leaching under the APPO conditions, as confirmed by the analysis of the post-reaction aqueous product which showed undetectable Zr⁴⁺ ions using inductively coupled plasma (ICP) testing. The crystalline structure of the spent m-ZrO₂ catalyst was confirmed to be substantially unchanged by XRD analysis.

The amount of the heterogeneous oxidation catalysts may vary and can be based relative to the amount of biomass. For example, a weight ratio between the heterogeneous oxidation catalyst and the biomass may be present in a range from about 1:20 to about 1:1.

According to another aspect of the invention, the efficiency of the heterogeneous oxidation catalyst to activate the reactive gas (e.g., oxygen) can be modified by treating a first metal oxide heterogeneous oxidation catalyst with a second metal oxide heterogeneous oxidation catalyst to provide a mixed oxide product that is suitable for the APPO process described herein. Exemplary mixed oxide catalysts include, but are not limited to, modified monoclinic zirconium oxides, such as a titanium zirconium mixed oxide, a cerium zirconium mixed oxide, a vanadium zirconium mixed oxide, a manganese zirconium mixed oxide, a cobalt zirconium mixed oxide, an iron zirconium mixed oxide, a ruthenium zirconium mixed oxide, a tungsten zirconium mixed oxide, a molybdenum zirconium mixed oxide, a rhenium zirconium mixed oxide, a magnesium zirconium mixed oxide, a calcium zirconium mixed oxide, or a potassium zirconium mixed oxide, any of which can be prepared by methods such as co-precipitation and impregnation. The modified mixed oxide heterogeneous catalysts can be dried (e.g., at 100° C.) overnight, and calcined (e.g., at 700° C. for 6 hours).

According to embodiments of the invention, the APPO reaction medium includes water, but water also participates as a reactant in the APPO reaction process by facilitating various hydrolysis steps. Co-solvents may be used, so long as the selected co-solvent does not substantially interfere with the APPO reaction process. Exemplary co-solvents include but are not limited to γ-valerolactone, n-heptane, methyltetrahydrofuran, tri-n-octylphosphine oxide, trioctylamine, toluene, and ionic liquids. The water, as well as any co-solvents, should be sufficiently pure so as to not detrimentally affect the APPO process. For example, potable water is sufficiently pure for the APPO process. The amount of water can be adjusted to facilitate acceptable levels of mass transfer during the mixing process. For example, the water can be present in a sufficient amount to provide the APPO reaction mixture having a weight percent of biomass in a range from about 1 wt % to about 25 wt %, based on the total weight of the APPO reaction mixture. In one embodiment, the weight percent of the biomass is in the range from about 2 wt % to about 20 wt %, about 2 wt % to about 15 wt %, about 3 wt % to about 10 wt %, or about 4 wt % to about 8 wt %. Accordingly, exemplary biomass weight percentages can be about 5 wt % or about 10 wt %.

According to embodiments of the invention, the APPO process is performed in the presence of a reactive gas. The APPO process can be performed under a pressurized atmosphere of the reactive gas at pressures equal to or greater than atmospheric pressure and/or at temperatures equal to or greater than room temperature. According to one embodiment, the reactive gas includes oxygen. Accordingly, the APPO process may utilize air, which advantageously includes about 21% by volume of oxygen. According to another embodiment, the oxygen is present in an initial proportion in a range from about 0.05% to about 100%, which is based on its partial pressure of a total reactive gas pressure. For example, the initial proportion may be in the range from about 1% to about 50%, from about 1% to about 21%, from about 1% to about 10%, from about 20% to 80%, from about 30% to about 70%; or from about 40% to about 60%. Further, the APPO process may be performed under a pressurized atmosphere of the reactive gas, wherein an initial pressure prior to heating is in a range from about 15 pounds per square inch (psi) to about 600 psi (about 1 atmosphere (atm) to about 40 atm). In another example, the initial pressure of the reactive gas prior to heating may be in a range from about 200 psi to about 500 psi (about 13 atm to about 33 atm). The identity of levulinic acid and glycolic acid were determined by analysis using high performance liquid chromatography.

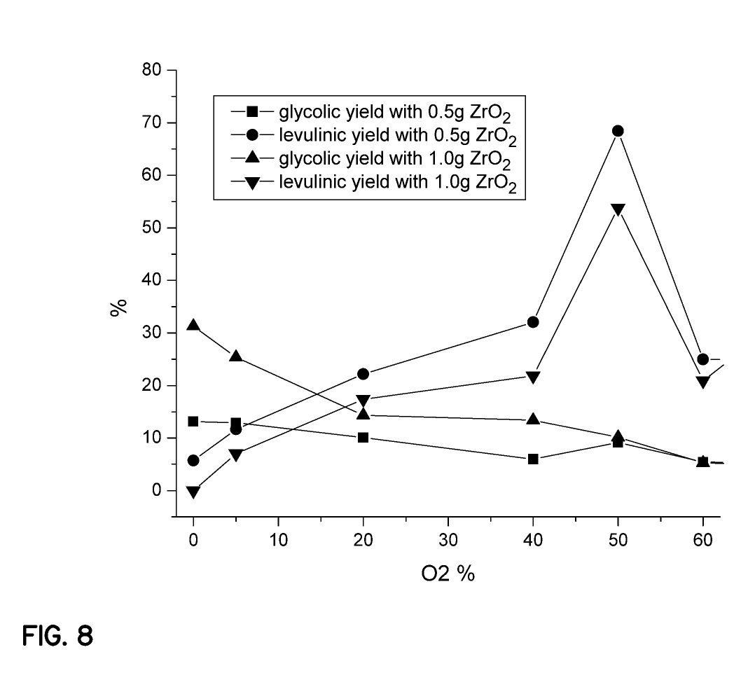

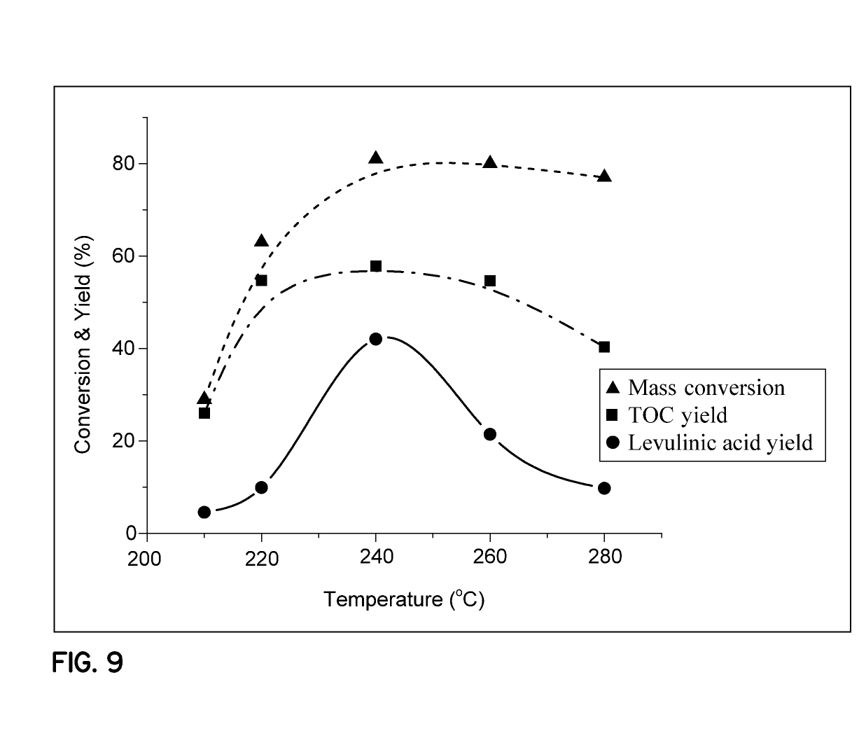

According to one aspect of the APPO reaction, the reactive gas (e.g., O₂) partial pressure can affect the resultant product distribution and/or yield of APPO processing of lignocellulosic biomass, as shown in FIG. 8, which is a graphical representation showing the effects of O₂ partial pressure on the APPO processing of xylan with ZrO₂ catalyst. With specific reference to FIG. 8, the O₂ partial pressure affects the xylan conversion to levulinic acid and glycolic acid over a ZrO₂ heterogeneous oxidation catalyst. It was observed that the levulinic acid yield increased from about 6% to about 68% when the O₂ partial pressure was increased from 0% to 50% with 0.5 g ZrO₂ loading, while the glycolic acid yield decreased from about 13% to about 4%. The yield of levulinic was lower with 1.0 g ZrO₂ than with 0.5 g ZrO₂ while the glycolic yield was higher with 1 g ZrO₂ than with 0.5 g ZrO₂.

The APPO process also may be performed at an elevated temperature, such as at a temperature that is in a range from about 100° C. to about 300° C. For example, the APPO may be performed at a temperature that is the range from about 150° C. to about 280° C., from about 160° C. to about 260° C., or from about 180° C. to about 240° C. With specific reference to FIG. 9, the temperature dependency of the catalytic partial oxidation of cellulose is demonstrated over a temperature range of 210° C. to 280° C. The effects of temperature on the conversion, total organic content (TOC) and levulinic acid yields of the catalytic partial oxidation of cellulose in aqueous phase is shown under the following reaction conditions: 240° C., 20 minutes, 2.8% O₂, 540 psi initial pressure, 9.1 wt % cellulose loading, 1:2 catalyst to cellulose mass ratio.

It should be further appreciated that the rate of increasing the temperature (ramp rate) of the APPO reaction mixture to the desired temperature can vary. For example, according to various embodiments, the APPO reaction mixture can be heated at a ramp rate of about 10° C./min, about 15° C./min, about 20° C./min, or about 25° C./min. Once the desired temperature is reached, the APPO reaction mixture can be maintained for a sufficient duration to permit the desired conversion of the biomass to the platform carboxylic acids to be effected. Subsequent to the reaction heating period, the temperature of the APPO reaction mixture can be decreased to stop (or substantially slow down) the oxidation reaction prior to subjecting the reaction mixture to further processing steps.

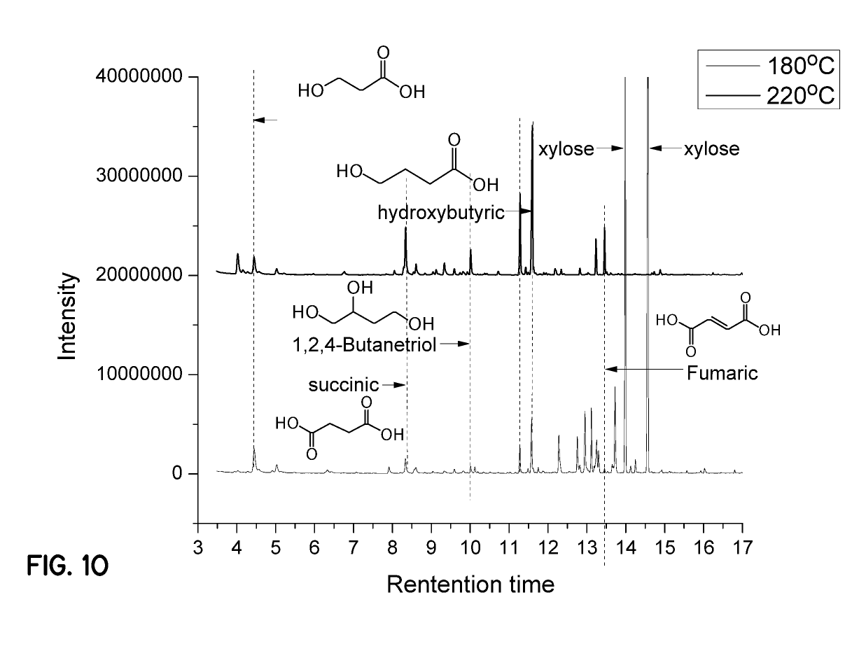

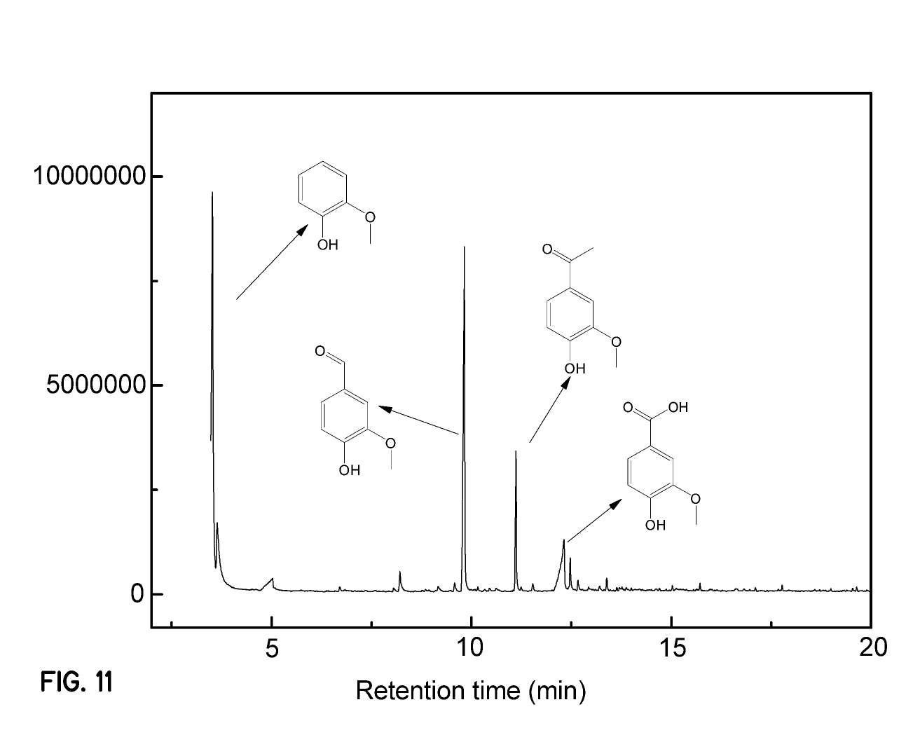

According to another aspect of the present invention, the APPO process can be conducted in one or more separate steps, which can be performed under different heating, catalyst, and/or reactive gas atmosphere conditions. For example, the APPO processing of lignocellulosic biomass can be processed in a single heating step within a desired temperature range (e.g., about 240° C. to about 260° C.), as described above. Alternatively, the APPO reaction mixture can be subjected to a first heating step whereat the mixture is heated to a first temperature. Thereafter, at least a portion of the reaction mixture can be subjected to a second heating step whereat the portion can be heated to a second temperature which is higher than the first temperature. In one example, the first temperature can be from about 180° C. to about 220° C. and the second temperature can be from about 230° C. to about 270° C. The first temperature can be sufficient to convert/oxidize hemicellulose to carboxylic acids, such as 3-hydroxyproprionic acid, 4-hydroxybutanoic acid, fumaric acid, and succinic acid, and alcohols such as 1,2,4-butanetriol, and also sufficient to convert/oxidize lignin to vanillin, vanillylmethylketone, vanillic acid, and guaiacolaren (see FIGS. 10 and 11, respectively). The second heating step can affect the conversion of cellulose to its APPO oxidation products, as discussed in more detail below. If desired, the oxidation products from the first heating step can be separated from an unreacted portion of the cellulose component by standard separation techniques (e.g., filtration) to provide a separate stream of platform compounds that can be upgraded to their respective hydrocarbons.

It should be further appreciated the batch-type APPO processes described herein can be readily adapted to flow cell-type applications.

The heterogeneous oxidation catalyst and insoluble materials, such as unreacted biomass, can be separated from the aqueous solution of platform carboxylic acids by standard physical separation techniques, such as filtration or centrifugation. It should be noted that under the appropriate conditions, lignocellulosic biomass can be oxidatively-deconstructed to provide monofunctional carboxylic acid(s), bifunctional hydroxyl carboxylic acid(s), dicarboxylic acid(s), or combinations thereof, in substantially higher yields relative to non-catalytic methods. For example, percent conversions of cellulose, lignin and corn stover through the APPO process with selected Au/ZnO catalysts and without catalyst are shown in Table 1. Experimentally, the initial materials, i.e., 10 g biomass, 20 g water, and 2 g catalyst, were loaded in a 100 mL Parr reactor and subjected to the following operation conditions: T=260° C.; Reaction Time=15 mins; Initial pressure of Air=500 psi. Percent conversions are based on ash-free dry mass of biomass.

TABLE 1
Conversions of cellulose, lignin and corn stover through the
APPO process with selected Au/ZnO catalysts and without catalyst

| | Au/ZnO | No Catalyst |
|---|---|---|
| Cellulose | 100% | 66% |
| Lignin | 90% | 47% |
| Corn Stover | 85% | 52% |

The mass balances of all experiments were in the range of 92% to 102%. In all cases, the APPO catalyst resulted in almost complete conversion of cellulose and 90% conversion of model lignin. The conversion of the as-received corn stover pellets was 85% at 260° C. and 15 min reaction time, which is superior to the hydro-liquefaction process under similar operation conditions. A few other studies using batch reactions of corn stover have also resulted in primarily producing organic acids, where the pH value of the aqueous phase decreased from about 7 to the range of about 3 to 4. The total organic carbon analysis found that the carbon yield in the aqueous phase products is ˜30-70%. While this carbon yield is comparable to the fermentation process of producing ethanol from glucose, these studies utilized much cheaper materials, cellulose and even raw lignocellulosic biomass, as feedstocks instead of using glucose or starch. Moreover, no hydrogen is needed in the APPO process. A short-term stability study using cellulose as the model feed found that the Au/ZnO catalyst was stable and the cellulose conversions were kept the same for three runs without regeneration.

To identify the carboxylic acid products produced from the oxidative deconstruction of cellulose using the APPO process over various catalysts, 10 g of cellulose, 20 g of water, and 2 g of catalysts were first loaded into a 100 mL Parr reactor. The APPO process was performed under the following operation conditions: T=200-260° C.; Reaction Time=15-30 minutes; Initial pressure of Air or lean air=400-500 psi. The conversions of cellulose to the liquid and gaseous products over the different catalysts were in the range of 60% to 00%.

After the reaction, the resultant aqueous phase samples were prepared for total organic carbon, HPLC, and GCMS analysis. Tables 2 and 3 below show the carbon yields of the aqueous phase products from the APPO processing of cellulose over the noble metal loaded metal oxide catalysts and the metal oxides catalysts, respectively, at 260° C. Two major carboxylic acids produced by the APPO process of cellulose were lactic acid and levulinic acid. The highest carbon yield of lactic acid, which is about 12%, was obtained over the 1% Au/W—ZrOx catalyst while the highest carbon yield of levulinic acid, 28%, was acquired over the 1% Pd/ZrO₂ catalyst under the specified operation conditions. The operation conditions, especially temperature, have a significant effect on the selectivity of the carboxylic acid. Table 4 below shows that, by lowering the temperature from 260° C. to 240° C. and keeping other operation conditions unchanged, the carbon yield of levulinic acid increased significantly from 13% to 43% over the 1% Au/ZrO₂ catalyst. Similarly, the levulinic acid yield increased from 23% to 40% over the ZrO₂ catalyst.

TABLE 2
Carboxylic acid products distribution produced by the APPO process of cellulose over
metal loaded metal oxide catalysts.

| Run | Catalyst | Total Organic Carbon | Lactic Acid | Levulinic Acid | Other Acids | Other Oxygenates |
|---|---|---|---|---|---|---|
| 1 | No Catalyst | 28.29% | 0.00% | 11.28% | 5.05% | 11.96% |
| 2 | 1% Au/ZSM-5 | 34.94% | 0.31% | 15.19% | 7.89% | 11.55% |
| 3 | 1% Au/Zeolite Y | 52.21% | 3.19% | 27.17% | 16.08% | 5.78% |
| 4 | 1% Au/α-Al2O3 | 31.09% | 0.41% | 9.20% | 3.45% | 18.02% |
| 5 | 1% Au/ZrO2-Hydrotalcite | 51.63% | 7.07% | 0.00% | 7.18% | 37.38% |
| 6 | 1% Au/Hydrotalcite | 55.09% | 7.52% | 0.00% | 11.42% | 36.15% |
| 7 | 1% Au/W—ZrOx | 57.30% | 12.13% | 10.19% | 5.36% | 39.61% |
| 8 | 1% Au/γ-Al2O3 | 54.51% | 9.04% | 2.35% | 5.13% | 38.00% |
| 9 | 1% Au/Ce—La—ZrOx | 49.23% | 8.97% | 5.97% | 6.69% | 27.60% |
| 10 | 1% Au/TiO2 | 46.52% | 4.50% | 5.92% | 4.71% | 31.39% |
| 11 | 1% Au/ZnO | 66.06% | 10.03% | 2.21% | 11.36% | 42.46% |
| 12 | 1% Au/ZrO2 | 51.64% | 8.40% | 13.27% | 4.84% | 25.13% |
| 13 | 1% Au/ZSM-5 | 37.65% | 2.02% | 20.39% | 11.56% | 3.68% |
| 14 | 1% Pd/Ce—La—ZrOx | 47.68% | 8.92% | 4.74% | 6.11% | 27.91% |
| 15 | 1% Pd/W—ZrOx | 47.06% | 5.70% | 6.43% | 5.06% | 29.87% |
| 16 | 1% Pd/ZrO2 | 49.28% | 8.39% | 27.62% | 4.69% | 8.58% |

Reaction conditions: Temperature: 260° C.;
Initial pressure 400 psi, N₂: 97.2%, O₂: 2.8%;
reaction time: 20 min.

TABLE 3
Carboxylic acid products distribution produced by the APPO process of cellulose over
metal oxide catalysts.

| Run | Catalyst | Total Organic Carbon | Lactic Acid | Levulinic Acid | Other Acids | Other Oxygenates |
|---|---|---|---|---|---|---|
| 17 | Zeolite-β | 44.24% | 0.79% | 18.04% | 6.61% | 18.80% |
| 18 | ZSM-5 | 39.22% | 0.34% | 14.04% | 6.08% | 18.77% |
| 19 | Zeolite-γ | 43.38% | 3.64% | 16.92% | 8.29% | 14.53% |
| 20 | ZnO | 58.99% | 11.53% | 0.43% | 7.04% | 39.99% |
| 21 | Hydrotalcite | 56.70% | 7.33% | 0.00% | 10.03% | 39.33% |
| 22 | γ-Al2O3 | 41.41% | 5.78% | 10.86% | 4.97% | 19.80% |
| 23 | α-Al2O3 | 32.09% | 0.03% | 7.91% | 6.31% | 17.84% |
| 24 | ZrO2 | 54.61% | 8.24% | 22.78% | 5.38% | 18.22% |
| 25 | Ce—La—ZrOx | 44.17% | 8.40% | 5.91% | 6.81% | 23.04% |
| 26 | La—Zr—Ox | 50.49% | 9.81% | 5.09% | 6.01% | 29.57% |
| 27 | W—ZrOx | 49.88% | 10.17% | 7.58% | 6.16% | 25.97% |
| 28 | ZrO2-Hydrotalcite | 58.97% | 8.00% | 0.00% | 8.33% | 42.64% |

Reaction conditions: Temperature: 260° C.;
Initial pressure 400 psi, N₂: 97.2% O₂: 2.8%;
reaction time: 20 min.

TABLE 4
High levulinic acid poduction by he APPO process of cellulose over Au/ZrO₂ and
ZrO₂ catalyts.

| Run | Catalyst | Total Organic Carbon | Lactic Acid | Levulinic Acid | Other Acids | Other Oxygenates |
|---|---|---|---|---|---|---|
| 29 | No Catalyst | 50.15% | 0.64% | 8.28% | 23.24% | 17.99% |
| 30 | 1% Au/ZrO2 | 56.96% | 7.82% | 42.55% | 6.58% | 0.00% |
| 31 | ZrO2 | 57.72% | 8.06% | 40.35% | 9.31% | 0.00% |

Reaction conditions: Temperature: 240° C.;
Initial pressure 400 psi, N₂: 97.2%, O₂: 2.8%;
reaction time: 20 min.

Other identified carboxylic acid products include formic acid, acetic acid, glycolic acid, succinic acid, propionic acid, and isobutyric acid. However, the yields of those acids are significantly lower than those of lactic acid and levulinic acid. There is a considerable amount of unidentified oxygenated water soluble organic compounds in the aqueous phase products over some specific catalysts. A qualitative GC/MS analysis found that other oxygenates include ketones, aldehydes, and a trace amount of sugars. By optimizing the operation conditions, these oxygenates can be further oxidized to acids. As such, according to one embodiment, the APPO process is selective for the production of lactic acid and levulinic acid, i.e., lactic acid and levulinic acid are the major products produced.

Table 5 below further compares the mass conversions, the total organic carbon (TOC) yields in aqueous products, and the yields of major carboxylic acid products from the APPO reaction process of cellulose with various heterogenous oxidation catalysts and under different process conditions. Reaction conditions: (100 mL Parr reactor; 2.0 g cellulose, 20.0 g water, and 1.0 g catalyst; initially charged with 350 psi 97.2% N₂+2.8% O₂; reaction time was 25 min). The mass balances were in the range of 98-101%. Without adding a solid catalyst, the major carboxylic acid products were levulinic acid, formic acid, and acetic acid, which were catalyzed by protons in high temperature water. Acidic heterogeneous oxidation catalysts, including zeolite β, zeolite Y, and ZSM-5, increased the yields of levulinic acid and formic acid. Hydrotalcite, a basic heterogeneous oxidation catalyst, completely suppressed the production of levulinic acid but catalyzed lactic acid formation, presumably by a base catalyzed retro-aldol condensation. ZrO₂ provided the highest yields of both levulinic and lactic acid. In one embodiment, a maximum levulinic acid yield of 52% (Table 5, Entry 7) was achieved from the APPO processing of cellulose over the ZrO₂ catalyst (surface area 117 m²/g) at 240° C. and 2.8% initial O₂ partial pressure. An average levulinic acid yield of 50.0±2.0% was obtained by repeating six experiments under the same conditions as those in Table 5, Entry 7. The initial biomass loading may affect the product yield as a decrease of levulinic acid yield from 52% to 42% was observed with increasing the cellulose loading from 4.8 wt % to 9.1 wt % (compare entries 6 and 7 of Table 5).

TABLE 5
Comparison of the mass conversions, the TOC yields, and the carbon molar yields of
major carboxylic acids of the APPO of cellulose with and without catalysts.

| | | Temp. | Mass | TOC | Carbon Yields of Exemplary Aqueous Products | | | | | |
|---|---|---|---|---|---|---|---|---|---|---|
| Entry | Catalyst | (° C.) | Conversion | Yield | Formic | Acetic | Glycolic | Lactic | Levulinic | Others |
| 1 | No catalyst | 260 | 59% | 28% | 1.8% | 1.2% | 1.3% | 0.0% | 10.6% | 14.2% |
| 2 | No catalyst | 240 | 72% | 50% | 1.7% | 1.7% | 2.4% | 0.6% | 7.8% | 37.5% |
| 3^{[a]} | 0.5 M H₂SO₄ | 150 | 74% | 66% | 8.4% | 0.1% | 0.0% | 0.0% | 39.7% | 27.2% |
| 4 | γ-Al₂O₃ | 240 | 75% | 43% | 2.1% | 0.5% | 1.5% | 5.7% | 15.5% | 3.8% |
| 5 | TiO₂ | 240 | 43% | 68% | 1.7% | 0.0% | 2.4% | 0.0% | 5.5% | 10.5% |
| 6 | ZrO₂ | 240 | 81% | 61% | 2.9% | 1.5% | 3.6% | 8.3% | 42.0% | 2.6% |
| 7^{[b]} | ZrO₂ | 240 | 87% | 67% | 3.2% | 0.0% | 3.6% | 9.0% | 51.9% | 2.5% |
| 8 | ZrO₂ | 260 | 80% | 55% | 2.2% | 1.4% | 2.0% | 8.1% | 27.8% | 13.5% |
| 9 | Zeolite β | 240 | 74% | 44% | 4.6% | 0.5% | 1.5% | 0.7% | 16.6% | 2.3% |
| 10 | ZSM-5 | 240 | 50% | 45% | 4.1% | 0.7% | 1.1% | 0.2% | 13.5% | 2.2% |
| 11 | Zeolite Y | 240 | 41% | 30% | 1.8% | 0.6% | 1.9% | 2.6% | 10.1% | 10.9% |
| 12 | Hydrotalcite | 240 | 24% | 29% | 2.4% | 0.8% | 2.1% | 3.6% | 0.0% | 1.1% |

^{[a]}reaction time was 6 hours;
^{[b]}cellulose and catalyst loading were 1.0 g and 0.05 g, respectively.

In addition, as shown in FIG. 10, various aqueous phase products of the APPO of xylan (a hemicellulose) with ZrO₂ as catalyst at 180° C. and 220° C., respectively, were identified using gas chromatography-mass spectrometry (GC/MS) analytical techniques. An aqueous mixture of 1 g of xylan and 0.5 g of ZrO₂ was heated for 60 min at the specified temperature under an atmosphere comprising 5% O₂, 350 psi initial pressure. Hydroxybutyric acid, succinic acid, fumaric acid, and hydroxypropopionic acid were the major carboxylic acids products produced by the APPO reaction of xylan.

And as shown in FIG. 11, various aqueous phase products of the APPO of lignin with 10% MgO modified ZrO₂ at 200° C. were identified using GC/MS analytical techniques. An aqueous mixture of 1 g lignin and 0.5 g MgO/ZrO₂ was heated for 60 min at 200° C. under an atmosphere comprising 50% O₂, 400 psi initial pressure. Vanillin, vanillylmethylketone, vanillic acid, and guaiacol were the major products produced by the APPO reaction of lignin.

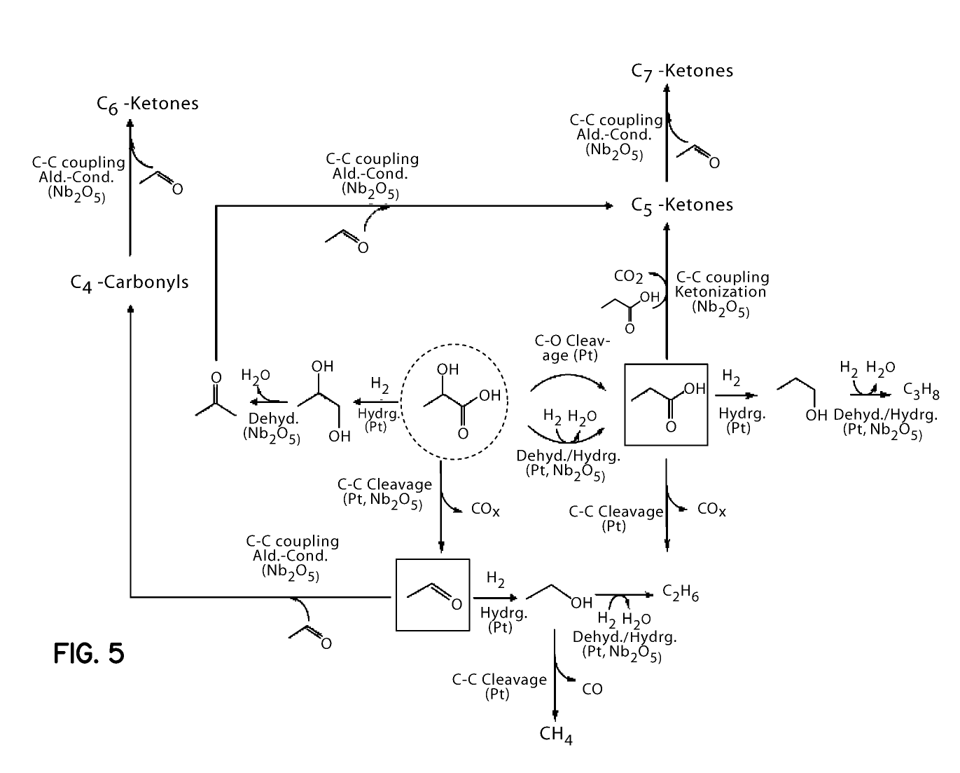

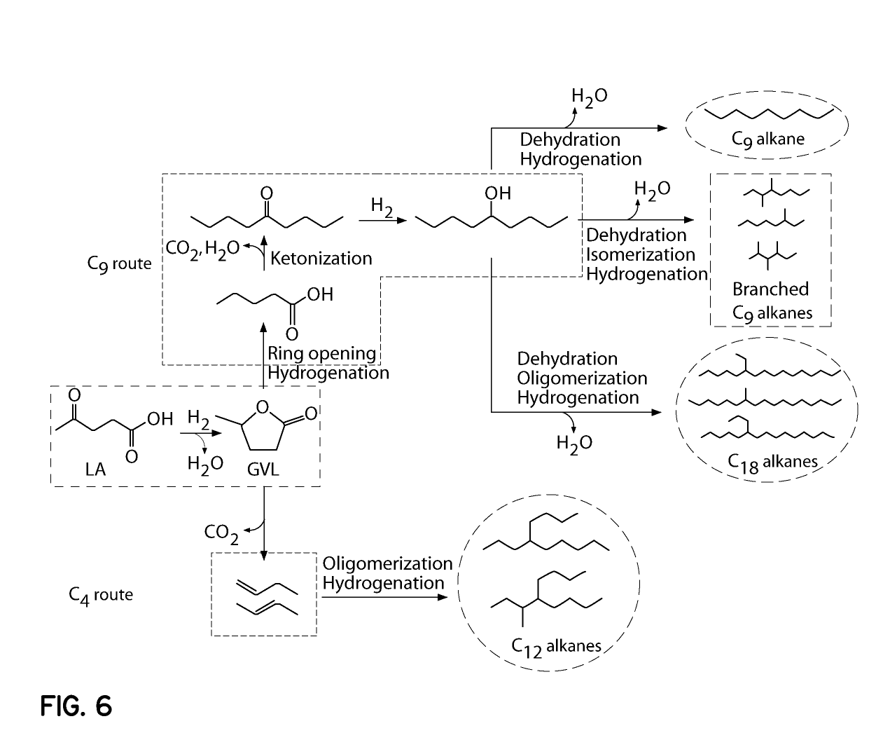

Several catalytic upgrading pathways are possible for upgrading mixed carboxylic acids to fuels and chemicals. For example, as shown in FIG. 3, ketonization of two acetic acids (C2) can produce one acetone (C3) followed by two complementary C—C coupling upgrading options. To the right, an aldol-condensation of two C3 ketones shows the formation of one C6 ketone. Conversely, to the left, the formation of one C3 alkene through a hydrogenation and a dehydration (i.e., a hydrodeoxygenation) of one C3 ketone followed by dimerization of two C3 alkenes to form one C6 alkene. As shown in FIG. 4, the hydrogenation of acetic acid (C2) can produce ethanol (C2), which can be followed by dehydration to form ethylene (C2). The C—C coupling upgrading pathway, for example, can include three ethylenes combining to form one C6 alkene. The dehydration/hydrogenation, and C—C coupling reactions, as shown in FIG. 5, outlines a reaction scheme for upgrading of lactic acid in dilute aqueous solutions (30 wt %) over 0.1% Pt/Nb₂O₅. Similarly, as shown in FIG. 6, an exemplary reaction scheme for upgrading levulinic acid to C4, C9, C12, and C18 alkanes is provided.

Exemplary ketonization catalysts include, but are not limited to, Ceria-Zirconia catalysts (Ce₁₋ₓZrₓO₂), manganesia-alumina (MnO₂—Al₂O₃) catalysts, or ruthenium/titania (Ru/TiO₂) catalysts. Exemplary aldol condensation catalysts include, but are not limited to, palladium/magnesia-alumina (Pd/MgO—Al₂O₃), palladium/zinc oxide-alumina (Pd/ZnO—Al₂O₃) palladium/hydrotalcite (Pd/Hydrotalcite) catalysts. Exemplary hydrodeoxygenation catalysts include, but are not limited to, Ni—Mo/Al₂O₃, Co—Mo/Al₂O₃, Mo₂C, WC, VN, Pt/Al₂O₃, Pd/Al₂O₃, etc. Thus, according to another aspect of the present invention, the APPO deconstructed biomass can be subjected to upgrading. Exemplary methods for upgrading are described in U.S. Patent Application Publication No. 2009/0255171, the contents of which are incorporated by reference herein in their entirety.

With reference again to FIG. 7 and according to another embodiment, an integrated biorefinery 100 for the conversion of biomass, such as lignocellulosic biomass, is provided. In the biorefinery 100, the biomass can be delivered to a holding tank 110 for processing prior to being introduced to an APPO reactor 120. Thereat, the processed biomass is mixed with water and one or more oxidation catalysts under a non-inert atmosphere (e.g., air) and heated to a desired reaction temperature (e.g., about 100° C. to about 400° C.). To enable the APPO reaction mixture to be heated above its boiling point, the APPO reactor 120 may be pressurized accordingly. By-products produced from the APPO process include a mixture of organic acids, along with variable amounts of carbon dioxide and some char. The aqueous reaction mixture can be subsequently transferred to a separation vessel 130, wherein a portion of the water is removed to concentrate the organic acids. Optionally, the concentrated (or dehydrated) organic acids can be transferred (e.g., by truck) to an upgrading reactor system 140 wherein ketonization reactions (e.g., in the presence of a ceria-zirconia catalyst) convert the carboxylic acids to ketones, which further undergo intermolecular aldol condensations (e.g., in the presence of a palladium/hydrotalcite catalyst) to build larger carbon-containing molecules. The reaction products of the upgrading reactor system 140 can be transferred to a phase separator system 150 to separate the upgraded intermediates from any unreacted organic acids prior to hydrodeoxygenating the upgraded intermediates. Separation can be achieved by techniques such as liquid-liquid extraction, membrane separation, distillation, or the like. The upgraded intermediates are transferred to a hydrodeoxygenation system 160, where treatment with hydrogen in the presence of a hydrodeoxygenation catalyst (e.g., a NiMo/Al₂O₃ catalyst) yields hydrocarbons (e.g., gasoline) and water.

The non-limiting examples of Tables 3-5 above, in accordance with various principles of the present invention, are discussed below.

### EXAMPLES

### Materials

The following reagents and products were used as received for the experiments: Cellulose Microcrystalline, average particle size 50 μm, D(+)-Glucose Reagent ACS Grade, and D(+)-Cellobiose, 98%, were purchased from Acros Organics. Gold 1% on zinc oxide granulate (AUROlite™ Au/ZnO), gold 1% on aluminum oxide extrudates (Aurolite™ Au/Al₂O₃), and gold 1% on titanium dioxide extrudates (Aurolite™ Au/TiO₂), were purchased from Strem Chemicals.

For chemical analysis and derivitization, the following reagents and products were used as received: Glacial Acetic Acid, Acrylic Acid 99% inhibited with 200 ppm MEHQ, BSTFA+TMCS, 99:1, Butyl Alcohol 99%, Butyl Lactate 98%, Butyl Acetate>99%, Citric Acid 99%, D-(+)-Glyceraldehyde>98%, Hydrochloric Acid 37% ACS grade, L-(+)-Lactic Acid 98%, DL-Malic Acid>99%, Oxalic Acid+99%, Propionic Acid>99.5%, Pyridine Anhydrous 99.8%, Pyruvaldehyde, 40% wt. Solution in water, Sulfuric Acid ACS reagent 95%, and Trifluouroacetic Acid (TFA) 99%, were purchased from Sigma Aldrich. Formic Acid 98% was purchased from Fluka. Levulinic Acid Butyl Ester 98% was purchased from TCI America.

The following reagents were used for catalyst preparation: aluminum oxide catalyst support (low surface area), hydrogen tetrachloroaurate (III) trihydrate (HAuCl₄.3H₂O), Zeolite-β, Zeolite-Y, Zeolite ZSM-5, and zirconium oxide catalyst support, were purchased from Alfa Aesar. Ammonium hydroxide was purchased from EMD. Synthetic hydrotalcite was purchased from Sigma Aldrich. Zinc oxide was purchased from Strem Chemicals. Water used during catalyst preparation and testing and chemical analysis was ultra-pure water (18.2 MΩ·cm at 25° C.) from EMD Millipore.

Preparation of Supported Gold Catalysts.

1 wt % gold catalysts supported on ZrO₂, ZSM-5, Zeolite β, Zeolite Y, and hydrotalcite were prepared using wet impregnation method and deposition-precipitation method. Briefly, the incipient wetness impregnation is as follows: an appropriate amount of a 0.0127 mol/l HAuCl₄.3H₂O solution was diluted and added on the support material. The wet support materials were dried overnight at 100° C. and then calcined at 550° C. for 3 hours in static air with a heating rate of 5° C./min. For the deposition-precipitation method, a 0.001 mol/l HAuCl₄.3H₂O solution was adjusted to pH 7 by dropwise adding concentrated NH₄OH solution, and was heated to 60° C. The support was added to the solution and vigorously stirred for 2 hours. Using vacuum filtration, the resultant precipitates were separated and washed with 750 ml deionized water. They were then dried at 110° C. for 8 hours, followed by calcination at 250° C. for 5 hours with a heating rate of 5° C./min.

Preparation of Zirconium Oxide (Zirconia) Catalysts

Monoclinic zirconium oxide (Alfa Aesar) was ground and calcined in air at 250° C. for three hours before using as the catalyst. Sulfated zirconium oxide and sodium hydroxide passivated zirconium oxide were prepared by wet impregnation. A solution of 0.87 M sulphuric acid was used for wet impregnation of zirconium oxide. The wetted zirconium oxide was dried at 100° C. for overnight, and then calcined in air in a box furnace at 550° C., with a 5° C./min heating ramp, for five hours. To impregnate zirconium oxide with sodium hydroxide, the zirconium oxide pellets were immersed in a solution of 0.1 M NaOH and then transferred into the round bottom flask of a rotovap. The pellets were dried under vacuum and at 100° C. in the rotovap for twelve hours. The NaOH impregnated ZrO₂ pellets were washed with water until the pH of wash water was 7, then dried at 100° C. overnight, and calcined in air at 250° C. for three hours.

ZrO₂ nanocrystal samples were synthesized at 200° C. under autogenous pressure for 20 h in a glass-lined stainless-steel autoclave containing solutions of urea (CO(NH₂)₂ and zirconyl nitrate (ZrO(NO₃)₂.xH₂O). Deionized water and methanol were used as solvents for synthesizing monoclinic ZrO₂ (m-ZrO₂) and tetragonal ZrO₂ (t-ZrO₂), respectively. The concentration of Zr⁴⁺ in the solutions was 0.4 M, and the urea/Zr⁴⁺ molar ratio was 10. The resulting precipitates were washed thoroughly with water and methanol, treated at 110° C. overnight in ambient air, and then calcined at 400° C. for 4 h in dry air and nitrogen for m-ZrO₂ and t-ZrO₂, respectively.

The synthesis of macroporous zirconium oxide was as follows: 150 mL water solution with a pH of 13.5 was made up by the addition of NH₄OH. 15 mL zirconium propoxide (70 wt % in 1-proponal) was added to the water solution. The solution was aged at room temperature for 24 hours and was then placed in a rotovap to dry under vacuum at 100° C. for five hours. The dried powders were washed with DI water until the pH was 7. After drying overnight at room temperature, the zirconium oxide powders were collected, ground and calcined in a tube furnace at 1000° C. for five hours with flowing air.

Modified zirconia catalyst was synthesized by two methods: co-precipitation (“C”) and impregnation (“I”). In method C (Co-precipitation method), a mixed solution of ZrOCl₂.8H₂O and another metal chloride precursor was adjusted to pH 10 using aqueous ammonia. The resulting precipitate was filtered, washed with deionized water, dried overnight at 373 K, and then calcined in air at 973 K for 4 h. With method I (Impregnation method), an aqueous solution of starting metal chloride precursor was added to ZrO₂ (pretreated at 250° C.), then dried overnight at 373 K, and calcined at 973K for 4 h. The modified ZrO₂ catalysts prepared were CrOₓ/ZrO₂, MnOₓ/ZrO₂, CuO/ZrO₂, WO/ZrO₂, CoO/ZrO₂, MoOₓ/ZrO₂, and VOₓ/ZrO₂, with 1 wt %, 5 wt %, 10 wt % of the modification metal species.

Catalyst Testing.

Reactions in a 100 mL stirred Parr microreactor were carried out by suspending the catalyst (1.0 g) in a solution of biomass (2.0 g) in water (20 ml). In each reaction, a glass liner, supplied by Parr Instrument, was used to minimize reactant contact with the metal reactor walls. 20 mL of deionized water was placed in the liner, along with desired amount of cellulose, generally 2 grams. The catalyst to be tested was ground in a mortar and pedestal and placed in the liner at the desired loading. The liner was placed inside the reactor, and the reactor was charged with the reactive gas to the desired initial pressure. For example, the reactor can be charged to 400 psi with 2.8% oxygen lean air (e.g., 97.2% N₂) prior to heating. The reactor was heated at a ramp rate of 10° C./minute until the desired set temperature was reached. During the reaction, mixing was achieved through an internal propeller operating at 1200 RPM. Once the set temperature was attained, the reactor was held at the set temperature for 15 to 30 minutes then quenched in an ice bath to quickly drop the temperature. The reactor was cooled to approximately 25° C., and then the gas pressure was recorded and vented. The reactor was immediately broken down and the solid residue remaining on the propeller and reactor head was recovered and dried for the calculation of mass conversion. The liner was removed and the aqueous and solid fractions were separated using a Büchner funnel with Whatman® 42 filter paper (2.5 micron particle retention) and a vacuum pump. The liquid portion weight was recorded and the filter, filter paper, and solid residues were dried over night at 110° C. The water lost through drying was calculated through subtraction of pre and post drying weights.

The additional non-limiting examples of Tables 6-11 below, in accordance with various principles of the present invention, describe performance testing of other various heterogeneous oxidation catalysts utilizing the methods disclosed in the Product Analysis section below.

Further yet, in order to test the performance of modified catalysts on cellulose conversion, CrOₓ/ZrO₂, MnOₓ/ZrO₂, CuO/ZrO₂, WO/ZrO₂, CoO/ZrO₂, MoOₓ/ZrO₂, and VOₓ/ZrO₂ catalysts were utilized under exemplary APPO conditions: 1 g cellulose, 0.5 g modified ZrO₂, 330 psi total initial pressure, 2.8% O₂ partial pressure, 240° C., and 30 min. Tables 6-11 below set forth the results of the APPO processing of cellulose using the various modified catalysts. In Table 6, the results of a copper oxide modified zirconia catalyst for the APPO oxidation of cellulose are provided and discussed below.

TABLE 6
Copper Oxide Modified Zirconia Catalyst

| Catalyst | Conv. | Error | TOC | Glycolic | Lactic | Acetic | Levulinic | Propionic | Acrylic | HMF | Total |
|---|---|---|---|---|---|---|---|---|---|---|---|
| ZrO₂ | 90% | 2.3% | 65% | 4.2% | 8.9% | 0.0% | 50.8% | 1.6% | 0.2% | 4.9% | 77.5% |
| 1% Cu/OZrO₂ C | 81% | 3.3% | 56% | 4.0% | 1.9% | 0.0% | 19.6% | 0.0% | 9.1% | 7.3% | 42.0% |
| 1% CuO/ZrO₂ I | 86% | 3.5% | 64% | 3.0% | 6.2% | 0.0% | 49.3% | 0.9% | 0.1% | 7.7% | 67.4% |
| 5% CuO/ZrO₂ I | 87% | 2.5% | 62% | 3.7% | 5.6% | 0.0% | 43.3% | 0.8% | 0.2% | 8.1% | 61.6% |
| 10% CuO/ZrO₂ I | 85% | 2.4% | 60% | 4.0% | 5.1% | 0.0% | 39.7% | 0.7% | 0.2% | 7.6% | 57.7% |

Using HPLC analysis, it was observed that changing the loading of the CuO in the modified CuO/ZrO₂ does not affect the cellulose conversion to any great extent. However, the levulinic acid yield decreases, but the hydroxymethylfurfural (HMF) yield increases. 1% CuO/ZrO₂ prepared by co-precipitation (denoted with “C”, whereas “I” signifies impregnation) shows much change in lactic acid, levulinic acid, and acrylic acid. From XRD results, it was observed that the crystalline structure of the modified catalyst does not change under the APPO conditions. Inductively Coupled Plasma (ICP) testing of the aqueous phase after the reaction of pure ZrO₂ is used as a catalyst, shows that only a small amount of Zr exists in the aqueous phase, i.e., about 0.38 ppm. And when 1% Cu/ZrO₂ (I) is used, there are 0.33 ppm Zr and 4.93 ppm Cu in the final liquid.

TABLE 7
Cobalt Oxide Modified Zirconia Catalyst

| Catalyst | Conv. | Error | TOC | Glycolic | Lactic | Acetic | Levulinic | Propionic | Acrylic | HMF | Total |
|---|---|---|---|---|---|---|---|---|---|---|---|
| ZrO₂ | 90% | 2.3% | 75% | 4.2% | 8.9% | 0.0% | 50.8% | 1.6% | 0.2% | 4.9% | 77.5% |
| 1% CoO/ZrO₂ C | 75% | 10.0% | 67% | 3.6% | 4.3% | 0.0% | 38.1% | 0.9% | 0.2% | 12.8% | 59.3% |
| 1% CoO/ZrO₂ I | 81% | 2.5% | 63% | 2.8% | 5.5% | 0.0% | 32.4% | 1.0% | 0.0% | 14.3% | 55.9% |
| 5% CoO/ZrO₂ I | 61% | 3.2% | 44% | 0.0% | 9.3% | 1.1% | 0.0% | 0.0% | 0.0% | 0.0% | 11.4% |
| 10% CoO/ZrO₂ I | 62% | −4.7% | — | 3.7% | 9.4% | 0.0% | 7.4% | 2.1% | 0.1% | 1.6% | 24.8% |

In Table 7 above, the results of a copper oxide modified zirconia catalyst for the APPO oxidation of cellulose are provided. From HPLC data, it can be observed that CoO/ZrO₂ changes the cellulose conversion. Catalyst with 1% loading of CoO results in about 35% levulinic acid yield, and much higher HMF yield. With 5% and 10% CoO/ZrO₂, new APPO products are evident by HPLC analysis. The ICP analysis of the aqueous phase after using 10% CoO/ZrO₂ provided 0.31 ppm Zr, 785 ppm Co.

TABLE 8
Chromium Oxide Modified Zirconia Catalyst

| Catalyst | Conv. | Error | TOC | Gluconic | Glycolic | Lactic | Adipic | Levulinic | Propionic | Acrylic | HMF | Total |
|---|---|---|---|---|---|---|---|---|---|---|---|---|
| ZrO₂ | 90% | 2.3% | 75% | 0 | 4.2% | 8.9% | 2.5% | 50.8% | 1.6% | 0.2% | 4.9% | 77.5% |
| 1% CrO₃/ZrO₂ I | 77% | 4.1% | 58% | 0.0% | 3.1% | 3.9% | 0.0% | 22.4% | 0.4% | 0.1% | 8.3% | 38.2% |
| 5% CrO₃/ZrO₂ I | 77% | 2.4% | 60% | 0.3% | 2.7% | 4.9% | 1.7% | 25.1% | 0.6% | 0.2% | 9.3% | 45.0% |
| 10% CrO₃/ZrO₂ I | 81% | 2.8% | — | 0.0% | 2.3% | 6.5% | 0.0% | 26.9% | 0.9% | 0.2% | 9.6% | 46.4% |

In Table 8 above, the results of a chromium oxide modified zirconia catalyst for the APPO oxidation of cellulose are provided. HPLC analysis revealed that CrOx/ZrO₂ does not provide much influence on cellulose conversion, but notably the levulinic acid yield decreases to 25%, while the HMF yield increases to 9%. The ICP analysis of the aqueous phase after using 1% CrO/ZrO₂ provided 0.3 ppm Zr, 0.076 ppm Cr.

TABLE 9
Manganese Oxide and Tungsten Oxide Modified Zirconia Catalyts

| Catalyst | Conv. | Error | TOC | Glycolic | Lactic | Acetic | Adipic | Levulinic | Propionic | Acrylic | HMF | Total |
|---|---|---|---|---|---|---|---|---|---|---|---|---|
| ZrO₂ | 90% | 2.3% | 75% | 4.2% | 8.9% | 0.0% | 2.5% | 50.8% | 1.6% | 0.2% | 4.9% | 77.5% |
| 1% MnOx/ZrO₂ I | 81% | 2.9% | 61% | 4.0% | 6.2% | 0.0% | 0.0% | 38.4% | 0.8% | 0.1% | 8.9% | 58.4% |
| 5% MnOx/ZrO₂ I | 64% | 1.7% | 53% | 3.2% | 6.0% | 0.0% | 1.9% | 18.6% | 1.2% | 0.2% | 8.8% | 39.8% |
| 1% WOx/ZrO₂ I | 89% | 2.4% | 60% | 4.2% | 5.4% | 0.0% | 0.0% | 39.1% | 0.7% | 0.1% | 6.4% | 55.9% |
| 5% WOx/ZrO₂ I | 73% | 1.5% | 50% | 6.8% | 3.7% | 3.4% | 0.0% | 43.9% | 1.1% | 0.1% | 0.0% | 58.9% |

In Table 9 above, the results of manganese oxide and tungsten oxide modified zirconia catalysts for the APPO oxidation of cellulose are provided. HPLC analysis revealed that WOx/ZrO₂ had little effect on the product distribution and the levulinic acid yield is maintained at a desirable level. Conversely, MnOx/ZrO₂ provided a higher HMF yield. Levulinic acid yield is maintained at 38% under 1% loading MnOx/ZrO₂. The ICP analysis of the aqueous phase after using 5% MnO/ZrO₂ provided 0.68 ppm Zr, 186.9 ppm. The ICP analysis of the aqueous phase after using 5% WO/ZrO₂ provided 0.29 ppm Zr, 148.7 ppm W.

TABLE 10
Vanadium Oxide and Molybdenum Oxide Modified Zirconia Catalysts

| | | | | | | 2 | | | | | | |
|---|---|---|---|---|---|---|---|---|---|---|---|---|
| | | | | | | hydroxyl | | | | | | |
| Catalyst | Conv. | Error | TOC | Glycolic | Lactic | butyric | Acetic | Levulinic | Propionic | Acrylic | HMF | Total |
| ZrO₂ | 90% | 2.3% | 75% | 4.2% | 8.9% | 0.6% | 0.0% | 50.8% | 1.6% | 0.2% | 4.9% | 77.5% |
| 1% VOx/ZrO₂ I | 82% | 3.0% | | 3.5% | 2.7% | 0.0% | 0.0% | 20.6% | 0.7% | 0.1% | 6.2% | 33.9% |
| 5% VOx/ZrO₂ I | / | / | | 4.0% | 4.2% | 0.0% | 1.2% | 15.9% | 0.3% | 0.1% | 6.1% | 32.0% |
| 1% MoOx/ZrO₂ I | 81% | 2.8% | 54% | 7.1% | 3.8% | 0.0% | 1.8% | 31.6% | 0.0% | 0.2% | 0.9% | 45.3% |
| 5% MoOx/ZrO₂ I | 75% | 1.4% | 49% | 9.5% | 5.7% | 1.7% | 2.6% | 38.5% | 0.0% | 0.2% | 1.7% | 62.1% |

In Table 10 above, the results of vanadium oxide and molybdenum oxide modified zirconia catalysts for the APPO oxidation of cellulose are provided. HPLC analysis revealed that MoOx/ZrO₂ lowers the both levulinic yield and HMF yield. Modifying VOx/ZrO₂ showed no major impact on the reaction distribution or yields. XRD analysis of the 5% MoOx/ZrO₂ catalyst showed different peak at 28 (20). The ICP analysis of the aqueous phase after using 1% MoO/ZrO₂ provided 0.3 ppm Zr, 2.07 ppm Mo. The ICP analysis of the aqueous phase after using 1% VO/ZrO₂ provided 0.25 ppm Zr, 0.058 ppm V.

TABLE 11
Nanosized Zirconia Catalyst

| Catalyst | Amount | | Conv. | Error | Succinic | Glycolic | Lactic | Formic | Acetic | Levulinic | Propionic | Acrylic | HMF | Total |
|---|---|---|---|---|---|---|---|---|---|---|---|---|---|---|
| ZrO₂ | 0.5 | g | 90% | 2.3% | 0.0% | 4.2% | 8.9% | 3.6% | 0.0% | 50.8% | 1.6% | 0.2% | 4.9% | 77.5% |
| ZrO₂ | 1 | g | 78% | 3.2% | 0.0% | 2.7% | 7.9% | 3.1% | 0.0% | 43.5% | 1.1% | 0.1% | 2.4% | 58.4% |
| Nano | 0.5 | | 72% | 4.3% | 0.1% | 3.1% | 1.4% | 4.1% | 0.0% | 16.3% | 1.4% | 0.2% | 10.0% | 36.5% |
| ZrO₂ | | | | | | | | | | | | | | |
| Nano | 1 | g | 72% | 1% | 0.3% | 9.9% | 1.4% | 7.5% | 4.6% | 12.8% | 1.4% | 0.1% | 1.5% | 36.7% |
| ZrO₂ | | | | | | | | | | | | | | |

In Table 11 above, the results of nano-sized zirconia catalysts for the APPO oxidation of cellulose are provided. HPLC analysis revealed that when nano-sized ZrO₂ was used as the APPO catalyst, the levulinic yield decreased. Different catalyst ratio shows the different products distribution. XRD analysis confirmed that the nano-sized ZrO2 maintained its monoclinic structure.

Product Analysis.

After the APPO reaction, the resultant aqueous phase product samples were prepared and tested: Total organic carbon (TOC) analysis, high performance liquid chromatography (HPLC), and gas chromatography coupling with mass spectrometer (GCMS) analysis. The gaseous products were analyzed using gas chromatogram equipped with thermal conductivity detector (TCD).

For total organic carbon analysis (TOC), the resultant aqueous phase was filtered through a 0.45 micron syringe filter then diluted 200 times with ultra-pure water. TOC was measured by a Shimadzu Total Organic Carbon Analyzer model TOC-V.

The aqueous products were analyzed by using a Shimadzu high performance liquid chromatography (HPLC). HPLC analysis was performed using a Shimadzu HPLC system equipped a UV-VIS Detector (Shimadzu SPD 10-AV) and Refractive Index Detector (Shimadzu RID-6A). For analysis of organic acids and reaction intermediates, the samples were separated in an Aminex 87-H column from Bio-Rad, using 5 mM H₂SO₄ as the mobile phase, 0.7 mL/min flow, at a column temperature of 55° C. For quantitative identification and results, the UV-VIS detector was utilized at 208 nm and/or 290 nm.

Derivatization of the polar components was performed in order to preform qualitative GCMS analysis and identification of unknown components in the aqueous phase. BSFTA with TMCS (99:1) was employed to methylate and silylate the carboxyl and hydroxyl functional groups in the polar components. 1 mL samples of the resultant aqueous phase were dried overnight in deactivated 1.5 mL Waters Maximum recovery vials. To the dried solids, 50 μL of acetonitrile was added and ultrasonicated for 1 hour to allow the solids to dissolve. After ultrasonication, 50 μL of pyridine and 150 μL of BSFTA with TMCS (99:1, Sigma) were added and the vials were capped. The capped vials were placed in a sand bath maintained at 65° C. for 2 hours to allow complete silylation. After silylation, the samples were cooled for 2 hours and 5 μL of the silylation mixture was diluted with 1.5 mL of acetonitrile. The samples were injected in an Agilent 6890 series GC/MS equipped with an Agilent DB5-SMS Column and Agilent 5973 Mass Selective Detector (TIC detector). The column temperature was maintained at 70° C. for 5 minutes then ramped at 10° C./min to 300° C. and held at 300° C. for 2 minutes.

After the reaction, the gaseous products were collected in a tedlar gas bag and analyzed using a SRI 8610C gas chromatogram equipped with 0.5 mL gas sampling loop and thermal conductivity detector (TCD). A Haysep D packed column was employed (6 ft. ⅛ in) with a flow rate of 11 ml/min helium. The heating profile employed allowed separation of H₂, N₂, CO, CH₄, and CO₂. The column temperature was initially held at 40° C. then increased to 200° C. at a heat rate of 50° C./min.

The post-reaction aqueous phase supernatant was filtered, and then diluted with water for approximately 200 times for elemental analysis, which was performed using a Varian Vista PRO Inductively Coupled Plasma Optical Emission Sectrometry (ICP-OES). A HNO₃ solution containing 0.5 ppm Y³⁺ and 1500 ppm Ce²⁺ was added into the sample for internal standardization and ioniziation buffer.

While the invention has been illustrated by the description of one or more embodiments thereof, and while the embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative product and/or method and examples shown and described. The various features of exemplary embodiments described herein may be used in any combination. Accordingly, departures may be made from such details without departing from the scope of the general inventive concept.

What is claimed is:

1. A process for converting a biomass into hydrocarbons, the process comprising:
a) reacting the biomass under aqueous phase partial oxidation conditions in the presence of a heterogeneous oxidation catalyst to convert greater than 60 weight percent of the biomass to one or more carboxylic acids;
b) upgrading the one or more carboxylic acids to hydrocarbons in the presence of one or more catalysts, the catalysts catalyzing a ketonization reaction, an aldol condensation reaction, a hydrodeoxygenation reaction, or combinations thereof; and
c) separating out hydrocarbons from the one or more catalysts.

2. The process of claim 1, wherein reacting the biomass under aqueous phase partial oxidation conditions comprises:
i) mixing the biomass and the heterogeneous oxidation catalyst in an aqueous media; and
ii) heating the mixture in the presence of a reactive gas.

3. The process of claim 2, wherein the reactive gas comprises oxygen.

4. The process of claim 3, wherein oxygen is present in an initial proportion in a range from about 0.05% to about 100%, based on partial pressures of a total reactive gas pressure.

5. The process of claim 4, wherein the initial proportion is in the range from about 1% to about 10%.

6. The process of claim 2, wherein heating the mixture includes heating the mixture under a pressurized atmosphere of the reactive gas, wherein an initial pressure prior to heating is in a range from about 15 psi to about 600 psi.

7. The process of claim 6, wherein the initial pressure prior to heating is in a range from about 200 psi to about 500 psi.

8. The process of claim 2, wherein heating the mixture includes heating the mixture to a temperature that is in a range from about 100° C. to about 300° C.

9. The process of claim 2, wherein heating the mixture includes heating the mixture to a temperature that is in the range from about 180° C. to about 260° C.

10. The process of claim 1, wherein the one or more carboxylic acids comprises a monofunctional carboxylic acid, a bifunctional hydroxyl carboxylic acid, a dicarboxylic acid, or combinations thereof.

11. The process of claim 1, wherein the one or more carboxylic acids is selected from the group consisting of acetic acid, glycolic acid, lactic acid, oxalic acid, levulinic acid, succinic acid, propionic acid, hydroxybutyric acid, and vanillic acid.

12. The process of claim 11, wherein levulinic acid is produced as a major product of the one or more carboxylic acids.

13. The process of claim 1, wherein the heterogeneous oxidation catalyst comprises atoms, salts or oxides of gold, zinc, zirconium, titanium, or combinations thereof.

14. The process of claim 1, wherein the heterogeneous oxidation catalyst comprises zirconium oxide or a modified zirconium oxide.

15. The process of claim 1, wherein the heterogeneous oxidation catalyst is monoclinic zirconium oxide or a modified monoclinic zirconium oxide.

16. The process of claim 15, wherein the modified monoclinic zirconium oxide is selected from the group consisting of: a titanium zirconium mixed oxide, a cerium zirconium mixed oxide, a vanadium zirconium mixed oxide, a manganese zirconium mixed oxide, a cobalt zirconium mixed oxide, an iron zirconium mixed oxide, a ruthenium zirconium mixed oxide, a tungsten zirconium mixed oxide, a molybdenum zirconium mixed oxide, a rhenium zirconium mixed oxide, a magnesium zirconium mixed oxide, a calcium zirconium mixed oxide, and a potassium zirconium mixed oxide.

17. The process of claim 13, wherein the heterogeneous oxidation catalyst further comprises a support material selected from zinc oxide, zirconium oxide, titanium dioxide, aluminum oxide, or combinations thereof.

18. The process of claim 1, wherein the heterogeneous oxidation catalyst comprises a zeolite or a mesoporous silicate.

19. A process for converting lignocellulosic biomass into hydrocarbons, the process comprising:
a) reacting the lignocellulosic biomass under aqueous phase partial oxidation conditions in the presence of a heterogeneous oxidation catalyst and a reactive gas to convert greater than 60 weight percent of the lignocellulosic biomass to a plurality of carboxylic acids, wherein the plurality of carboxylic acids includes at least levulinic acid or lactic acid;
b) upgrading the plurality of carboxylic acids to hydrocarbons in the presence of one or more catalysts, the catalysts catalyzing a ketonization reaction, an aldol condensation reaction, a hydrodeoxygenation reaction, or combinations thereof; and
c) separating out hydrocarbons from the one or more catalysts.

20. The process of claim 19, wherein the heterogeneous oxidation catalyst comprises atoms, salts or oxides of gold, zinc, zirconium, titanium, or combinations thereof; and wherein the reactive gas comprises oxygen in an initial proportion in a range from about 0.05% to about 100% of a total reactive gas volume.
